(19) United States
(12) Patent Application Publication
(10) Pub. No.: US 2021/0394180 A1
(43) Pub. Date: Dec. 23, 2021

(54) PARALLEL ELECTRODES SENSOR

(71) Applicant: Georgia Tech Research Corporation, Atlanta, GA (US)

(72) Inventors: Ali Fatih SARIOGLU, Atlanta, GA (US); Ruxiu LIU, Atlanta, GA (US)

(21) Appl. No.: 17/292,333

(22) PCT Filed: Nov. 8, 2019
(86) PCT No.: PCT/US2019/060510

§ 371 Date: May 7, 2021

Related U.S. Application Data
(60) Provisional application No. 62/757,533, filed on Nov. 8, 2018.

(51) Int. Cl. B01L 3/00; G01N 15/12; G03F 7/00

(52) U.S. Cl. CPC B01L 3/502715; G01N 15/1218; G01N 33/48735; B01L 3/502707; G01N 2015/1254; G03F 7/0015

(57) ABSTRACT

Systems and methods to integrate electrical sensors comprising parallel electrodes into microfluidic devices that are manufactured using soft lithography are disclosed herein. With minimal fabrication complexity, more uniform electric fields than conventional coplanar electrodes are produced. The methods disclosed are also more suitable for the construction of complex electrical sensor networks in microfluidic devices due to greater layout flexibility and provide improved sensitivity over conventional coplanar electrodes.

### REFERENCE TO RELATED APPLICATION

This application claims priority to U.S. Provisional Application No. 62/757,533, filed on Nov. 8, 2018, which is relied upon and incorporated in its entirety by reference.

### STATEMENT REGARDING FEDERALLY SPONSORED RESEARCH OR DEVELOPMENT

This invention was made with government support under Award Nos. 1610995 and 1752170 awarded by the National Science Foundation. The government has certain rights in the invention.

### BACKGROUND

Electrical sensors, such as resistive pulse sensors, also known as Coulter sensors, are commonly integrated into microfluidic devices through micro-machined surface electrodes to create integrated systems for applications such as counting, sizing, and characterizing (including electrically) of micro/nanoparticles suspended in fluid. These types of sensors rely on modulation of impedance when a particle suspended in an electrolyte basses between two oppositely charged electrodes. In most of these implementations, electrodes are placed on the floor of microfluidic channels in a coplanar arrangement using a self-aligned fabrication process compatible with soft lithography. However, constricting electrodes to a plane not only leads to non-uniform electric fields affecting the sensor performance, but also complicates the design and scaling of multiplexed electrical sensor networks (e.g., the Microfluidic CODES system), by requiring the routing of three different coplanar electrodes on the same plane leading to excessively long traces with high electrical resistance.

Electrodes in parallel form can solve some of these issues, but their fabrication can be complex. For example, building Coulter sensors with parallel-electrodes in microfluidic devices typically rely on forming a grass-polyimide-glass sandwich structure. Such a structure not only involves a complex fabrication process that requires a critical alignment between layers, but also lacks the benefits of soft-lithography processes.

Therefore, there is a need for simple and robust fabrication methods, compatible with soft lithography, to create parallel-electrode sensors in microfluidic devices.

### SUMMARY

Parallel electrode sensors for use with microfluidic devices are described herein. An example parallel electrode sensor may comprise a first electrode, a second electrode, a substrate and a microchannel. The electrodes may be singular or may comprise a plurality of sets of electrodes. The electrodes may comprise a metallic layer. The substrate may have an upper surface upon which the first electrode may be deposited. The upper surface of the substrate may be lithographically patterned, for example by applying a photoresist, to form a pattern before the electrode is deposited thereon. The pattern may comprise an array. The microchannel may comprise an interior surface and the second electrode may be deposited on the interior surface of the microchannel. The interior surface of the microchannel may include side walls and a ceiling. The microchannel may be oriented above and bonded to the substrate to form a fluidic channel so that the interior surface of the microchannel and the upper surface of the substrate are facing each other, placing the first and second electrodes parallel to one another. The microchannel may be formed of a biocompatible material.

In another aspect, the invention is directed at a method of forming a parallel electrode sensor for use with microfluidic devices including lithographically patterning a first set of electrodes on an upper surface of a substrate, depositing a second set of electrodes on an interior surface of a microchannel, and bonding the microchannel with the substrate to form a fluidic channel with the first and second sets of electrodes being in parallel relation to one another. The microchannel can be formed from a biocompatible polymer molded a mold to have a ceiling and side walls. In some aspects, the mold can include a pattern, on which a metallic layer may be deposited to form the second set of electrodes. The metallic layer may be deposited by sputtering, with excess metallic layering can be removed to prevent short circuiting. The microchannel can be formed to have an electric port. The lithographically patterning of the first set of electrodes includes applying another metallic layer on the upper surface of the substrate after a lithographic pattern is applied to the upper surface. In some aspects, the pattern of the upper surface of the substrate and the imprinted pattern of the microchannel substantially match one another.

### BRIEF DESCRIPTION OF THE DRAWINGS

The details of the present disclosure, both as to its structure and operation, can best be understood by referring to the accompanying drawings, in which like reference numbers and designations refer to like elements.

FIGS. 1-9 illustrate exemplary parallel electrode sensors for use with microfluidic devices according to an aspect of the present disclosure.

FIG. 10 illustrates simulated electrical field distributions generated by different sensors according an aspect of the present disclosure.

FIG. 11A illustrates simulated electrical current modulation in response to cells in microfluidic channels integrated with different electrode configurations according to an aspect of the present disclosure.

FIG. 11B illustrates the ratio of simulated current modulation between a parallel-electrode and a coplanar-electrode sensor according to an aspect of the present disclosure.

FIG. 11C illustrates simulated electrical current modulation in microfluidic channels integrated with blanket electrodes with and without sidewall electrodes according to an aspect of the present disclosure.

FIG. 12 is a scanning electron micrograph of a PDMS microfluidic channel whose inner walls are coated with a metal layer according to an aspect of the present disclosure.

FIG. 13 illustrates simulated electric fields for the blanket electrodes with and without sidewall extensions according to an aspect of the present disclosure.

FIG. 14 is a schematic representation of a fabrication process for creating parallel electrodes in a microfluidic device according to an aspect of the present disclosure.

FIG. 15A illustrates a fabricated microfluidic device with fluidic and electrical connections according to an aspect of the present disclosure.

FIG. 15B is a schematic of the parallel electrode sensor of FIG. 15A.

FIG. 15C is a graph showing recorded sensor signals from the parallel electrode sensor of FIG. 15A with an exploded view of the signals at 0.04 s according to an aspect of the present disclosure.

FIG. 16A illustrates a fabricated microfluidic device with a coplanar-electrode sensor and a parallel-electrode sensor formed along the same microfluidic channel according to an aspect of the present disclosure.

FIG. 16B is a microscopic image of the sensors of FIG. 16A.

FIG. 17 illustrates resistive pulses recorded from the sensors of FIG. 16A-B.

FIG. 18 illustrates an exemplary microfluidic CODES device formed by parallel electrodes with sensors encoded with orthogonal digital codes fabricated according to an aspect of the present disclosure.

FIG. 19 illustrates representative signals corresponding with the sensors of FIG. 19 according to an aspect of the present disclosure.

### DETAILED DESCRIPTION

Systems and methods for fabricating parallel-electrode sensors in microfluidic devices that are compatible with soft lithography are disclosed herein. In an aspect, the present invention is directed towards a first set of electrodes that are placed in parallel relationship with a second set of electrodes within a microfluidic channel. In such aspects, the electrodes can be arranged within a microchannel and substrate, with a first electrode being placed within the microchannel, and the counter-electrode on a glass substrate, utilizing soft-lithography. With minimal fabrication complexity, the present systems and methods produce more uniform electric fields than conventional coplanar electrodes, and are also more suitable for the construction of complex electrical sensor networks in microfluidic devices due to greater layout flexibility. On one plane (e.g., a substrate in microfluidic device), at most two electrodes can be arranged freely. If there is a third electrode, the third electrode has to be placed according to the other two electrodes geometrically. In such instances, the arrangement of the third electrode is difficult, and sometimes even impossible. When another plane is presented in a parallel structure, the third electrode has the freedom to be arranged according the demand, providing more layout flexibility comparing to coplanar structure.

A metal film/metallic layer may be selectively deposited onto the inner surface of a microchannel as one of the sensor electrodes. This metal-coated microchannel may be bonded to a glass substrate containing micro-patterned surface electrodes to form parallel-electrode sensors. The electrode covering the microfluidic channel is conceptually analogous to ground planes in printed circuit boards and significantly simplifies the sensor network layout. Microfluidic devices may be integrated with sensors as formed as above to be used as Coulter sensors/resistive pulse sensors in counting and characterizing micro/nanoparticles suspended in fluids. In some aspects, the parallel-electrode sensors formed in the manner as discussed above can provide as much as 5x sensitivity improvement over conventional coplanar electrodes. This sensors modulate impedance when a particle suspended in an electrolyte passes between two oppositely charged electrodes.

As discussed above, in most microfluidic devices utilize micro-machined coplanar electrodes on the floor of microfluidic channels rather than utilizing the full channel geometry. While coplanar electrodes have a simple fabrication process compatible with the soft lithography, they generate non-uniform electric field affecting sensor performance and have complicated designs in large-scale multiplexed electrical sensor networks (e.g., Microfluidic CODES sensors) because of multiple electrode traces on a plane.

The invention is directed at the use of counter-facing parallel electrodes in microfluidic channels manufactured as discussed above while utilizing soft lithography process. Typical parallel electrode construction is very complex, with each device being fabricated separately. Therefore, most prior art systems have utilized coplanar construction. The present systems and methods provide a soft lithography-compatible fabrication method to create parallel-electrode Coulter sensors in microfluidic devices. In an aspect, one of the electrodes of the Coulter sensor is formed by a thin metal film blanket-deposited on the inner walls of a microfluidic channel via a soft lithography, while the other electrodes are lithographically patterned on the substrate according to the desired arrangement.

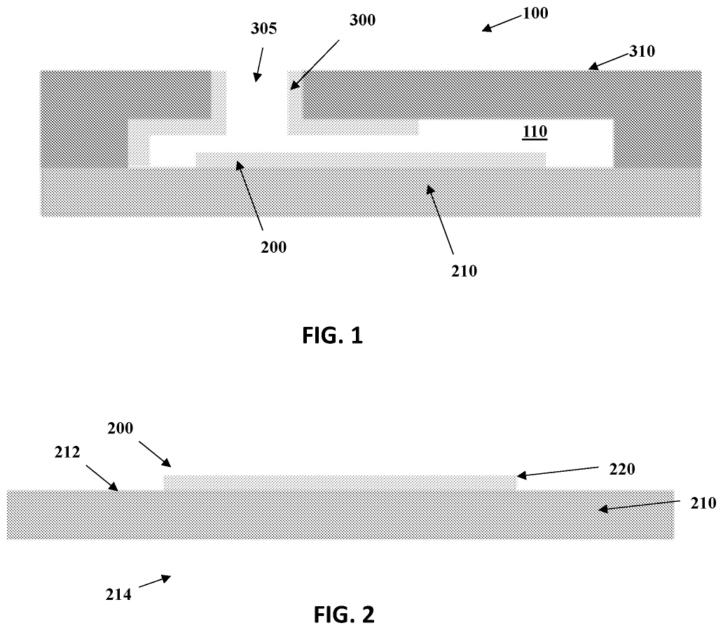

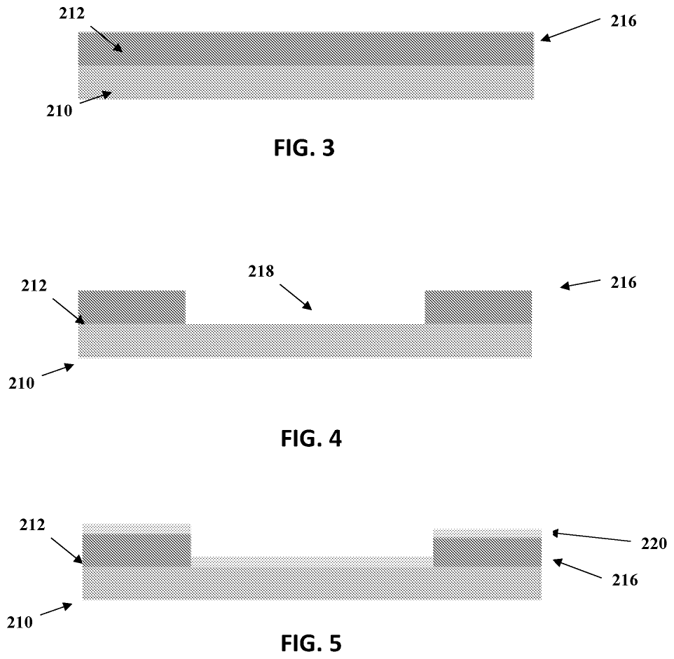

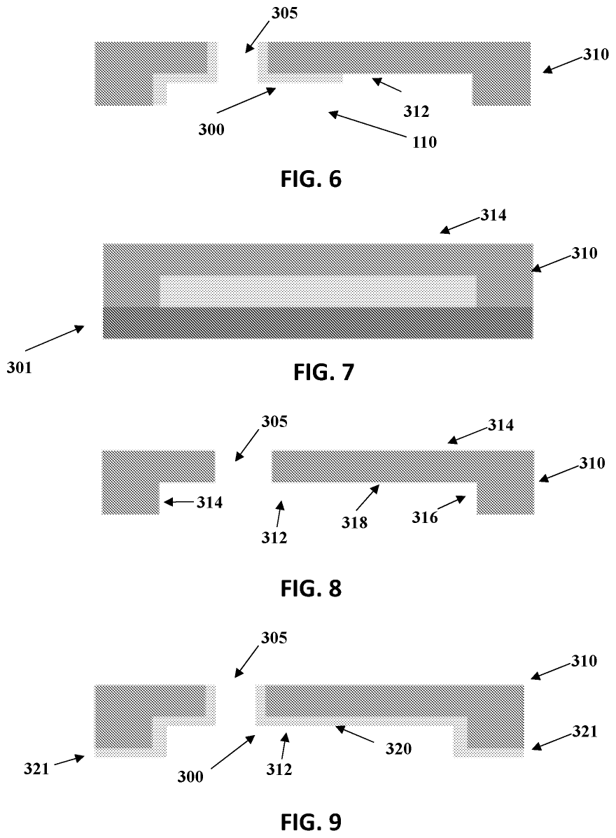

In an aspect, the current invention is directed at a parallel electrode sensor 100 for use with microfluidic devices. The parallel electrode sensor 100 includes a first set of electrodes 200 and a second set of electrodes 300, as shown in FIGS. 1-9. In an aspect, the first set of electrodes 200 is oriented to face the second set of electrodes 300. In such aspects, the first set of electrodes 200 can be positioned on a substrate 210 with the second set of electrodes featured on a microchannel 310. In an exemplary aspect, the substrate 210 and the microchannel 310 form a fluidic channel 110 through which fluid can flow, with the first and second set of electrodes 200, 300 configured to operate as a parallel electrode sensor 100 to monitor fluid passing through the fluidic channel 110. In an aspect, the parallel electrode sensor 100 is configured to monitor particles suspended in a fluid. “Particle” can refer to any particulate matter that can be contained in a liquid. Particles can include, but are is not limited to, cells, organelles, virus particles, nucleic acids, proteins, peptides, bacteria, worms (e.g., C. Elegans), exosomes, vesicles, pollen, sieved drug fractions, polymers, copolymers, silicon, glass, metal, metal oxide nanopartiples, or any combination of two or more components shown above. The size of such particles can range between 1 mm to 1 nm.

In an aspect, the first set of electrodes 200 is configured to be placed on a substrate 210. In an aspect, the substrate 210 is a glass substrate 210. However, in other aspects, the substrate can be of any material that is compatible to bond with the microchannel 310 and can be deposited with metals. Such materials include, but are not limited to, silicon-based materials (including glass), plastic, polymers, and the like. The glass substrate 210 includes an upper surface 212 and a lower surface 214.

The upper surface 212 of glass substrate 210 can be lithographically treated to receive the first set of electrodes 200. In an aspect, the substrate 210 is lithographically treated with a pattern that substantially matches the layout of the electrode(s) 200. In an aspect, the pattern can include an array. In an exemplary aspect, the pattern utilized on the substrate 210 can match a pattern implanted on the microchannel 310. In other aspects, the pattern can be any electrode structure needed. For example, single electrode with regular or non-regular shape, multiple electrodes encoded or non-encoded with different sequences can be used. No pattern needs to be avoided. In an aspect, the pattern is a single rectangular electrode pattern. In other aspects, a pattern for a Microfluidic CODES structure multiplexed electrode is used.

In an aspect, the pattern can be generated by using a photoresist. In such aspects, a photoresist 216 can be spun and patterned on the upper surface 212 of the glass substrate 210. A positive or negative photoresist 216 can be used. The photoresist 216 can be applied via an optical lithography process, producing a pattern 218 on the surface 212.

After the photoresist 216 is applied, a metallic layer 220 can be applied, with the metallic layer 220 covering the remaining photoresist and pattern on the upper surface 212 of the glass substrate 210. In an aspect, the metal of the metallic layer 220 is conductive and anti-corrosive. In some aspects, the metal can be biocompatible. The metallic layer 220 can be applied as a thin metal film. In such aspects, the metal film can have a thickness of 50 nm to 500 nm. In an aspect, the metallic layer 220 can include gold. In such aspects, a stack including gold (e.g., 20/80 Cr/Au) can be utilized. However, in other aspects, any common electrode metal, including, but not limited to, gold, silver, and platinum can be used. In aspects in which stacks are used, the adhesive layer (Cr in a Cr/Au stack) can include, but is not limited to, Cr, Ti, and the like.

In an aspect, the metallic layer 220 is deposited on the substrate 210. The metallic layer 220 can be deposited in various ways, including, but not limiting to, evaporation, sputtering, spun, and other commonly known methods. In an aspect, a 20/80 nm Cr/Au is evaporated to be applied to the substrate 210. After applied, the substrate is subjected to a process to remove the photoresist 216 (e.g., acetone bath), leaving the metallic layer 220 on the upper surface 212 on the substrate 210 to form the first set of electrodes 200.

In an aspect, the microchannel 310 is formed from a polymer. In an exemplary aspect, the polymer is selected from a biocompatible polymer. In some such aspects, polydimethylsiloxane (PDMS) can be used. However, other polymers can be used. In other aspects, other materials, including, but not limited to, gels, organic monolayers, and the like can be utilized to form the microchannel 310. In other aspects, the microchannel 310 can be formed of a composite of various materials. In an aspect, the microchannel 310 can be formed from a film, or other forms of extruded polymers. In other aspects, the thickness of the microchannel can range between 10 to 100 μm. In exemplary aspects, the thickness can range between 15 μm to 25 μm. While the thickness can vary, the dimensions should be on the same scale of the particle size that is to be monitored, so it can range from mm to nm.

In an aspect, the polymer is placed within a mold to form the microchannel 310. The microchannel 310 is formed to an interior surface 312 and an exterior surface 314. In an exemplary aspect, the microchannel 310 is molded, using a mold 301, to form sidewalls 314, 316, and a ceiling 318, which, in combination, form the majority of the fluidic channel 110 of the sensor 100. In an aspect, the microchannel 310 can be formed so that the fluidic channel 110 has a width of approximately 35 μm, and a height ranging between 14 μm to 65 μm. In other aspects, the fluidic channel 110 can widths and heights of various dimensions. In another aspect, the width and height of the fluidic channel 110 can be based upon the size of the suspended materials in the fluid.

In an aspect, the mold is patterned with photolithography, imprinting the same pattern on the interior surface 312 of the microchannel 310 (i.e., soft-lithography). The pattern on the mold and imprinted on the interior surface 310 of the microchannel 312 is selected for the ultimate determination of the layout of the second set of electrodes 300. Once the second microchannel 310 has been formed, the microchannel 310 can have ports 305 inserted. In such aspects, inlet, outlet, and electrical ports can be provided. While soft-lithography is preferred to be used for the formation of the microchannel, other known fabrication techniques used to shape plastics can be used.

In an aspect, the second set of electrodes 300 is formed from a metallic layer 320 deposited on the interior surface 312 of the microchannel 300. In an aspect, the metallic layer 320 is a thin metallic layer 320. In such aspects, when deposited, the metallic layer 320 has a thickness ranging between 50 nm to 500 nm. In an exemplary aspect, the metallic layer 320 has a thickness of approximately 500 nm. In an aspect, the metallic layer 320 is made of a metal similar to that used in the substrate 210, and is highly conductive and anti-corrosive. In addition, the metallic layer 320 is formed from a metal that can sputtered. Such metals can include, but are not limited to, gold, platinum, silver, and the like. In an exemplary aspect, the second set of electrodes 300 is formed from a metallic layer 320 of gold approximately 500 nm thick applied via sputtering on the interior surface 312. In an exemplary aspect, the metallic layer 320 is deposited on the pattern imprinted through the photolithography on the mold. In an aspect, metallic layer 320 is applied to only the ceiling 318 of the microchannel 310 along the inner surface 312. In such aspects, evaporation depositing and sputtering can work. In other aspects, the metallic layer 320 is applied to both the ceiling 318 and the side walls 314, 316. In such aspects, sputtering can be used.

While the application of metal 220, 320 to the substrate 210 and microchannel 310 respectively has been focused on their use as electrodes for use in a parallel electrode sensor, the methods of depositing of metals on these components can be utilized for other purposes as well. For example, the metals 220, 320 can be used to assist in functionality and chemical interactions of the surfaces of microchannels 310 and substrates 210 for other uses. surfaces in their functionality.

In an aspect, once the metallic layer 320 has been deposited on the interior surface 312 of the microchannel 310, the interior surface 312 is treated to prevent short circuits from the second set of electrodes 300 by removing excessive amounts 321 of the metallic layer 320. In an exemplary aspect, the interior surface 312 is treated by selectively removing the unnecessary metal. For example, when the microchannel 310 has been imprinted with a pattern, sticky tape can be applied to remove all the metal not found on the pattern.

After both the substrate 210 and the microchannel 310 have been treated to form the first set of electrodes 200 and the second set of electrodes 300, the substrate 210 and microchannel 310 are bonded together to form the sensor 100. The substrate 210 and microchannel 310 are bonded together so that the first set of electrodes 200 are aligned in parallel with a portion of the second set of electrodes 300. In an aspect, in applications in which the substrate 210 and the microchannel 310 are relatively small (e.g., microscale), a microscope can be used to assist with the alignment. In an aspect, once the substrate 210 and microchannel 310 are bonded. In an aspect, oxygen plasma can be used to treat the microchannel 310 and substrate 210 to bond them together, but other means can be used. Vacuums and/or clamps can be used to seal the device directly. After the bonding occurs, the inlet, outlet, and electric ports can be connected to a fluids and a power supply respectively. In an aspect, the electrodes 300 on the microchannel 310 are connected to a power source through a wire inserted through the electrical port, and the electrodes 200 on the substrate 210 are connected via contact pads. Once connected, the interior, that is the fluidic channel 110, is activated.

### Finite Element Analysis

Finite element analysis may be used to simulate the electric field distribution within the microfluidic channel generated by different types of sensors and calculate impedance variations for different particles. The performance of parallel electrodes and coplanar-electrode Coulter sensors is compared by fabricating the sensors on the same microfluidic platform and using cell suspensions to verify simulation results.

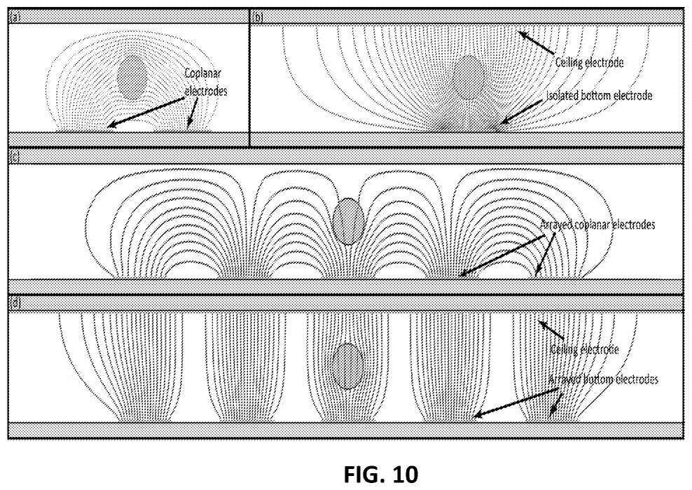

FIG. 10 shows a simulated electrical field distribution of a coplanar-electrode sensor and that of a parallel-electrode sensor fabricated according to an aspect of the present systems and methods. FIG. 10 shows a simulated electrical field distribution generated by an isolated coplanar sensor (a), parallel electrode sensors (b), arrayed coplanar electrode sensors (c) and arrayed parallel electrode sensors (d). COMSOL Multiphysics v5.3 AC/DC module was used to simulate Coulter sensor operation with coplanar or parallel electrode configurations and compare their performance. An electric field distribution was simulated within a microfluidic channel for different electrode configurations as illustrated in FIG. 1. Electric field distributions due to a single pair of coplanar electrodes (a) and an electrode paired with a parallel blanket electrode (b) were compared. While the parallel electrode configuration (b) provides a more uniform electric field throughout the height of the microfluidic channel, it also leads to a broadened non-uniform field distribution due to fringing effects due to the size mismatch between ceiling and bottom electrodes. This fringing effect could partially be alleviated by arraying bottom electrodes, which limits the broadening of the field for the inner electrodes (d) and provides a more uniform electrical distribution compared to arrayed coplanar electrodes (c).

To quantitatively analyze the sensor performance, the electrical current flow in the microfluidic channel was calculated and compared the amplitude of electrical current modulation in response to particles flowing between the electrodes. For these calculations, phosphate buffer saline (PBS) was assumed as the electrolyte and human cells as suspended particles. Corresponding electrical parameters used in the simulations for modeling particle-electrode interaction are summarized in Table 1 below:

TABLE 1
Parameters used in computer simulations

| Parameter | Value |
|---|---|
| Media conductivity | 1.4 S m⁻¹ |
| Media relative permittivity | 80 |
| Cytoplasm conductivity | 0.5 S m⁻¹ |
| Cytoplasm relative permittivity | 60 |

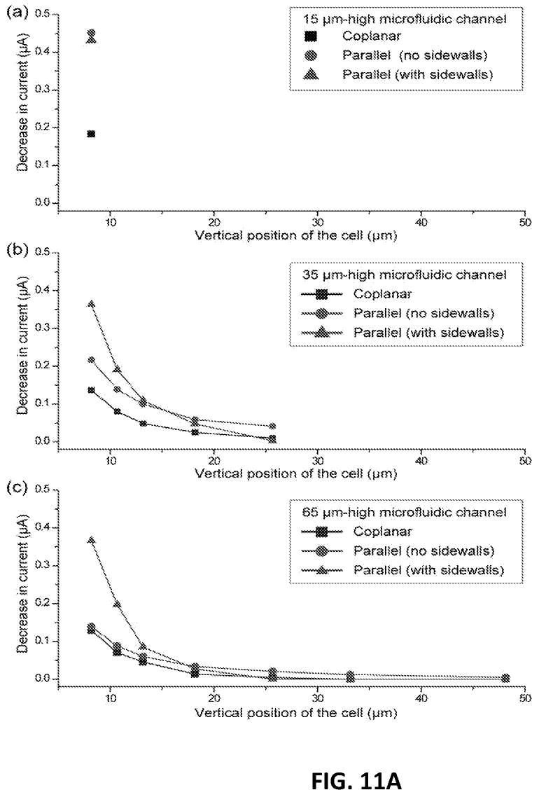

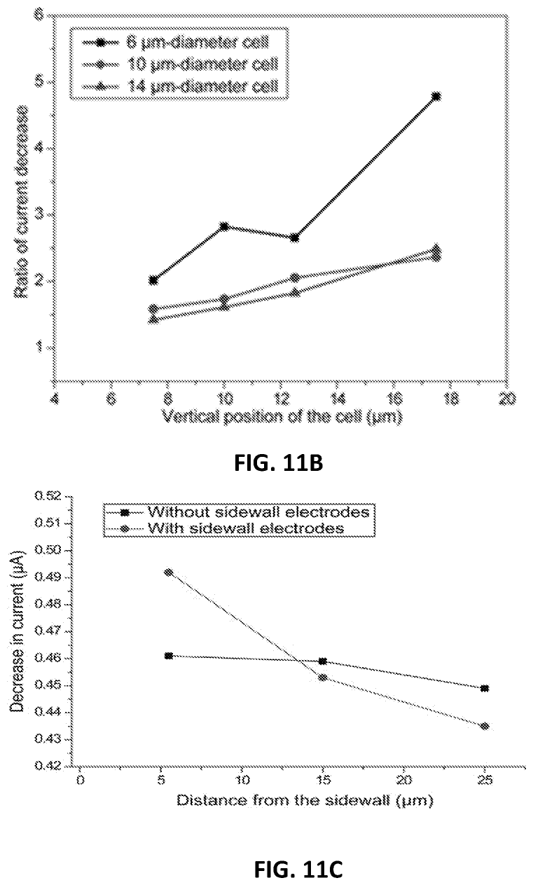

FIG. 11A illustrates simulated electrical current modulation in response to cells in microfluidic channels integrated with different electrode configurations. The decrease in electrical current as a measure of the sensor sensitivity for a 10 μm-diameter cell at multiple vertical positions in a 30 μm-wide microfluidic channel that is 15 μm- (a), 35 μm- (b), or 65 μm-high (c) was simulated. Three different electrode configurations were considered: a coplanar electrode pair (rectangle), a surface electrode in parallel to a larger ceiling electrode (circle), and a surface electrode paired with an electrode that covers both the ceiling and the sidewalls of the microfluidic channel (triangle). Because the ceiling electrode extends over the sidewalls of the microfluidic channel, sidewalls need to be considered especially for a narrow microfluidic channel. In 3D simulations, it was assumed that the cells were flowing in the central streamline (i.e., center positioned 15 μm from each sidewall). The current modulation for the parallel-electrode sensor was discovered to be higher than the coplanar-electrode sensor for the same particle. The enhancement in sensitivity was more pronounced for cells at elevated vertical positions due to the electrical field being confined to the floor of the microfluidic channel for coplanar electrodes. It was also observed that the extension of the ceiling electrode over the sidewalls increased the parallel-electrode sensitivity when a cell was positioned below a crossover elevation.

The effect of the cell size on the sensitivity of coplanar- and parallel-electrode sensors was also investigated. FIG. 11B illustrates the ratio of the simulated current modulation between a parallel-electrode and a coplanar-electrode sensor. The decrease in electrical current for the parallel- and coplanar-electrode configurations were compared in a 35 μm-high and 30 μm-wide microfluidic channel for 6 μm-, 10 μm-, and 14 μm-diameter cells. As shown in FIG. 2B, the simulations demonstrate that sensitivity enhancement is higher for smaller cells (2˜5× for 6 μm diameter cell) than larger cells (1.5˜2× for 14 μm-diameter cell). In addition, the effect of the cell size on the sensitivity enhancement was found to be generally higher for cells at higher elevations in the microfluidic channel.

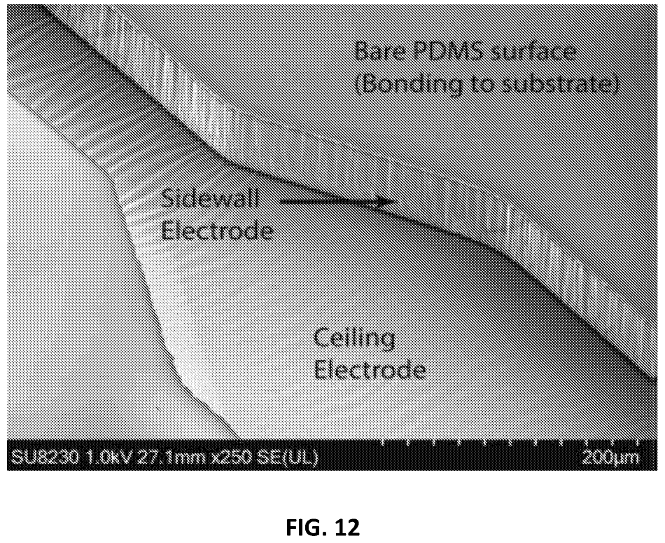

The effect of cell transverse position in the microfluidic channel on the current modulation amplitude for blanket electrodes with or without sidewalls was investigated. FIG. 12 illustrates an electron micrograph of a PDMS microfluidic channel whose inner walls are coated with a 500 nm-thick metal layer. Because the sputtering results in conformal deposition, the blanket electrode inevitably extends from the ceiling to the sidewalls of the microfluidic channel. Here, finite element analysis was employed to investigate the effect of the electrode-covered sidewalls on the performance of a Coulter sensor consisting of a blanket electrode.

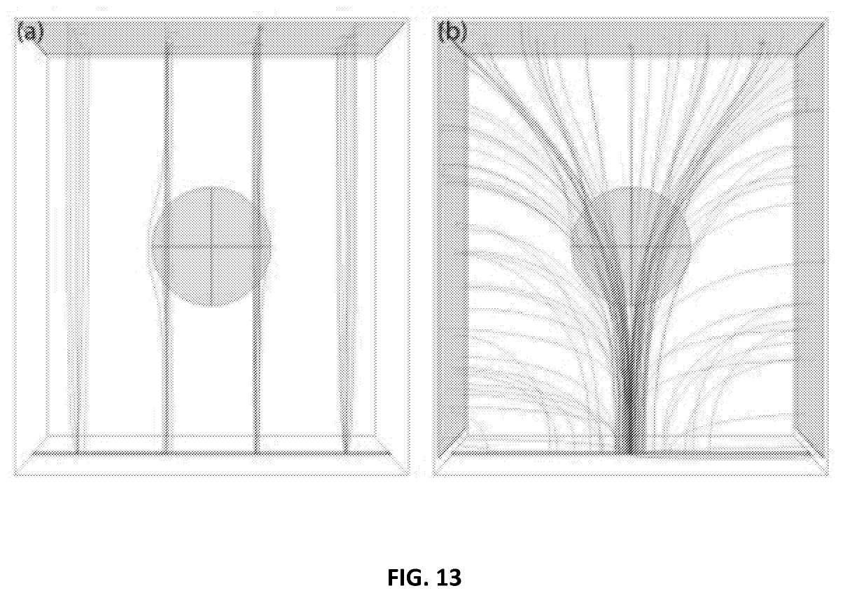

FIG. 13 show simulated electric fields for (a) a blanket electrode without sidewall extension and (b) a blanket electrode with sidewall extension. The field of view shows the cross-section of the microfluidic channel in both cases. It was discovered that the effect of transverse cell position on current modulation is more pronounced if sidewalls are electrically active and also that cells closer to the sidewalls produce larger current modulation.

As shown in FIG. 13, electrically active sidewalls were observed to provide alternative conduction paths, decreasing the overall impedance of the sensing region. COMSOL Multiphysics AC/DC module was used to perform the steady-state analysis of current flow between the electrodes and it was found that the background currents (no particle in the sensing region) for the sensors extending over sidewalls were at least 25% higher than the ones without the sidewall extension. Taken together, sidewall electrodes in Coulter sensors may enhance utility and can be used to optimize the design of electrical sensors in microfluidic systems for higher sensitivity.

FIG. 11C illustrates simulated electrical current modulation in response to the particles at different transverse positions in the microfluid channels integrated with the blanket electrodes with and without sidewall electrodes. Current modulation amplitude for the blanket-electrode sensor was found to be higher than the coplanar-electrode configuration for the same particle. The extension of the ceiling electrode over the sidewalls increased the blanket-electrode sensitivity only when a particle was positioned below a crossover elevation.

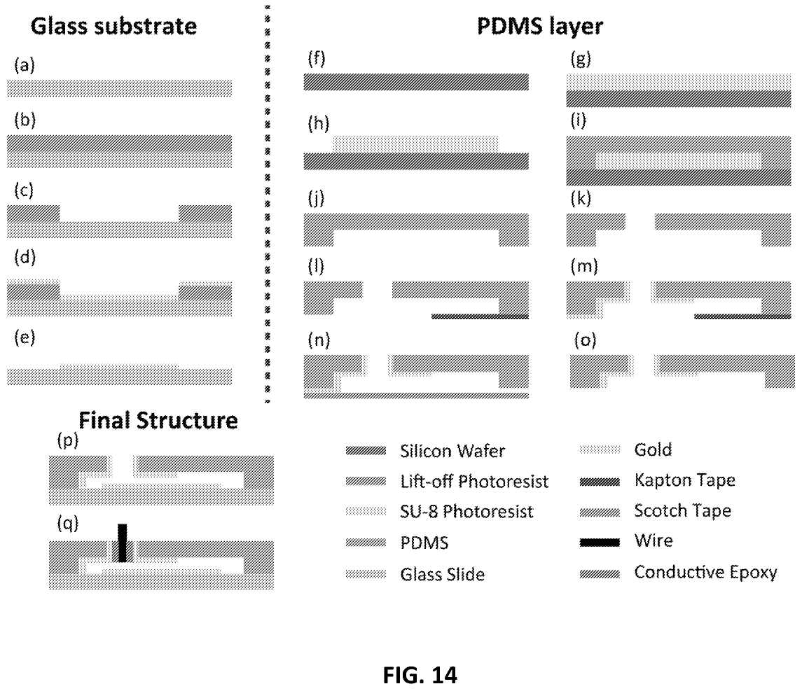

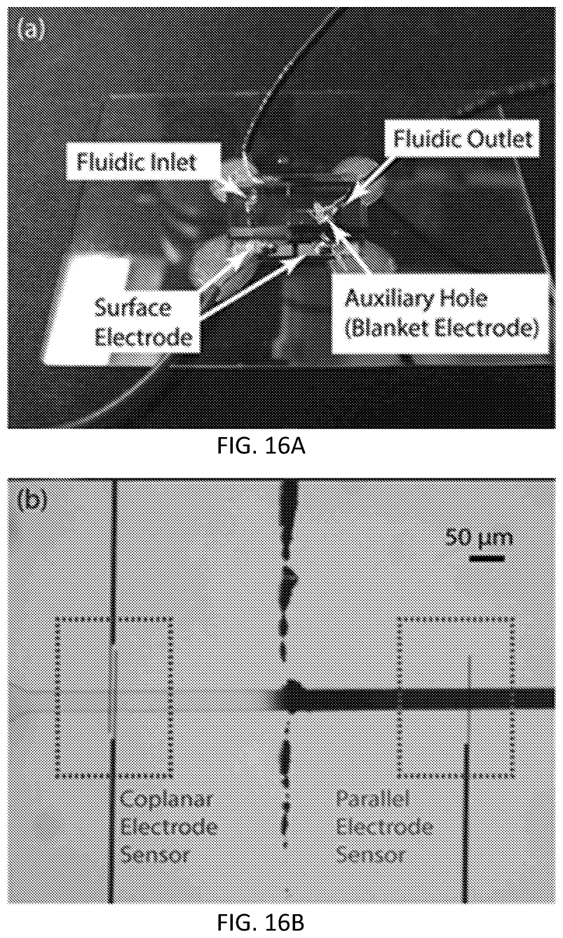

FIG. 14 shows schematics illustrating exemplary fabrication processes for creating parallel electrodes in microfluidic devices. In steps (a)-(e), a glass substrate with micropatterned surface electrodes is fabricated. In steps (f)-(o), the metal-coated PDMS microchannel is fabricated. Finally in steps (p)-(q), the two components may be bonded to form the final device. These are similar to the processes discussed above, and was followed to generate the device shown in FIG. 16A.

In an exemplary embodiment, the process may start the pattern of an SU-8 photoresist on a silicon wafer using photolithography to create a mold. Next, PDMS polymer is poured on the mold, degassed and baked. Then, the cured PDMS layer is peeled from the mold and holes are created using a biopsy punch to form fluidic inlet and outlet as well as an electrical port. A PDMS microchannel is then coated with gold via sputtering and transferred onto an adhesive tape to selectively remove the gold on the PDMS surface to prevent short circuits. Next, electrodes are fabricated on a glass substrate using a lift-off process. Photoresist is patterned on a glass slide with photolithography, evaporated using a Cr/Au film stack, and the resist is etched in acetone. Gold-coated PDMS microchannel and glass substrate with micro-patterned electrodes are then activated in oxygen plasma, aligned and bonded. Finally, a conductive epoxy-coated wire from the punched electrical port is injected.

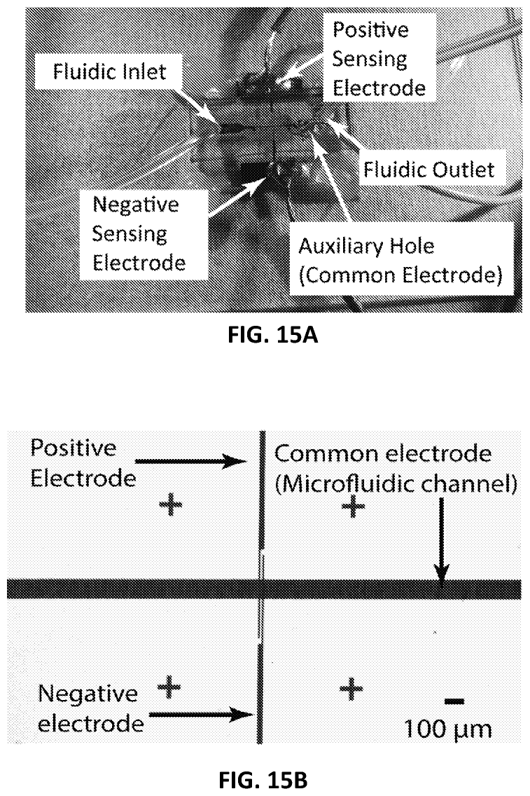

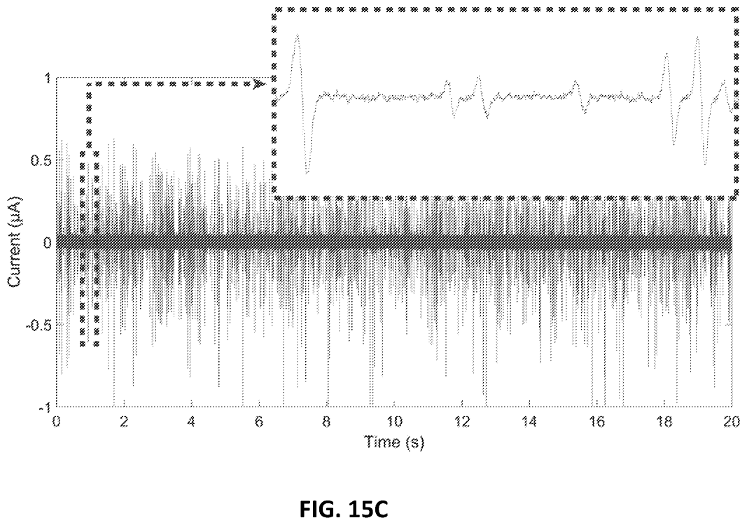

FIG. 15A shows an exemplary microfluidic device fabricated according to the present systems and methods comprising electrical and fluidic connections with 100 nm-thick electrodes. FIG. 15B is a schematic of the parallel electrode sensor of the device of FIG. 15A. To test the device, cultured human cancer cells were driven and suspended in buffer into the device using a syringe pump. The electrodes were driven with a 500 kHz sine wave and the root-mean-square (RMS) current amplitude was measured with a lock-in amplifier. FIG. 15C shows a graph of the test results in which approximately 2500 cancer cells were detected by the sensor during a 20 second period. An exploded view of the signal at 0.04 s shows that 7 cells were detected.

To experimentally compare the performance of coplanar- and parallel-electrode Coulter sensors, a microfluidic device that integrates two sensors based on coplanar electrodes and parallel electrodes along the same microfluidic channel was fabricated as shown in FIG. 16A.

FIG. 16B shows a microscopic image of the sensors of FIG. 16A formed along the same microfluidic channel. On the left side of the microfluidic channel, two 5 μm-wide coplanar electrodes separated by a 5 μm gap were created on a glass substrate. On the right side of the microfluidic channel, the inner walls of the microfluidic channel were coated with a gold film and only a single 5 μm-wide surface electrode was created on the glass substrate as shown in FIG. 16B. Because the same particle flowing in the microfluidic channel sequentially interacts with both sensors, this experimental platform allows for a direct comparison of signals from the two sensors.

As shown, the device consists of a glass substrate with micropatterned gold electrodes fabricated using a lift-off process and a polydimethylsiloxane (PDMS) microfluidic channel fabricated with a soft lithography process. A thin layer of negative photoresist was spun and patterned on a glass wafer using an optical lithography process, followed by the evaporation of a 20/80 nm Cr/Au stack. The wafer was then transferred to an acetone bath to remove the non-patterned region and diced into individual chips. The PDMS layer was created from a 15 μm-thick SU-8 mold patterned with photolithography. After the fluidic inlet, outlet and the electrical auxiliary holes were created using a biopsy punch, the inner walls of the PDMS microchannel was coated with a 500 nm-thick gold film with sputtering as coplanar-electrode-sensor-side (left side in FIG. 16B) of the microfluidic channel was masked with a Kapton tape. Next, the coated PDMS substrate was transferred onto a sticky tape to selectively remove the gold sputtered on the surface to prevent short circuits while leaving the gold within the microfluidic channel intact. Next, the PDMS and glass substrates were activated in oxygen plasma, aligned under a microscope and bonded. Finally, a conductive epoxy-coated wire from the auxiliary electrical port was injected to form an electrical connection to the blanket electrode and created the final device.

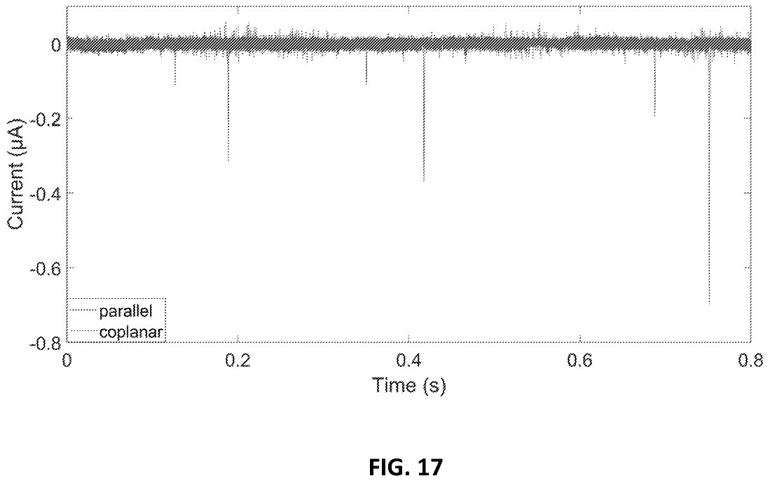

FIG. 17 illustrates electrical current modulation showing resistive pulses in response to the same cell recorded from a coplanar-electrode and a parallel-electrode sensor sequentially placed on the same microfluidic path. The microfluidic channel is 15 μm-high and 30 μm-wide. Cultured human breast cancer cells (MDA-MB-231) suspended in PBS were used as a sample to test the devices. The sample was driven through the microfluidic device using a syringe pump at a flow rate of 100 μL/h. To measure the change in electrical impedance of the microfluidic channel in response to flowing cells, both coplanar- and planar-electrode sensors were excited with 500 kHz sine wave and the resulting electrical current flows were measured. In the measurement setup, both sensors were excited with the same AC signal from the common electrode (i.e., the surface electrode for the coplanar electrode sensor and the blanket electrode for the parallel electrode sensor), and the current from the surface electrode acquired for each sensor. The current signals from both sensors were first converted into voltage signals using trans-impedance amplifiers, and then measured using a lock-in amplifier (Zurich Instruments HF2LI). The two sensor output waveforms containing signals for ˜200 cells were compared and it was found that the current modulation amplitude was 2˜4× higher for the parallel-electrode sensor compared to the coplanar-electrode sensor for the same cell as illustrated in FIG. 17. These results correspond with the simulation results.

Based on both computer simulation results and experiments with cell suspensions, it was discovered that the parallel-electrode Coulter sensor yields a higher sensitivity than the coplanar-electrode Coulter sensor, and the sensitivity enhancement is a function of the cell size, elevation, and microfluidic channel geometry.

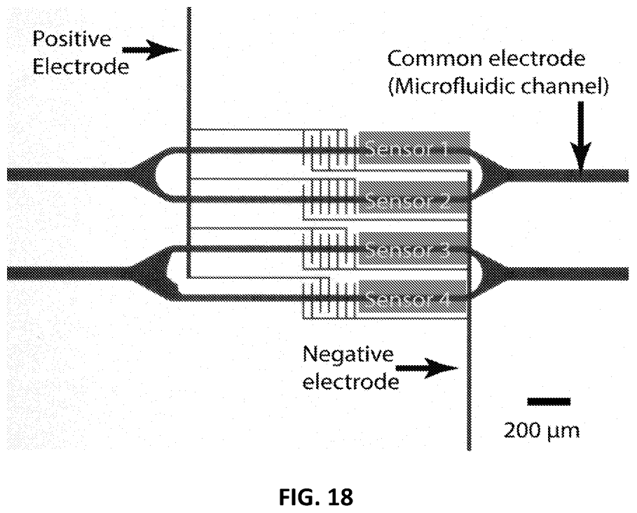

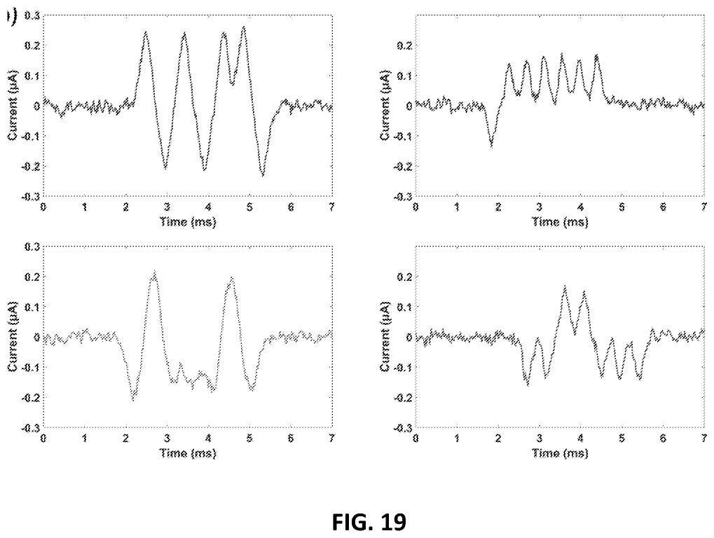

FIG. 18 shows a schematic of an exemplary Microfluidic CODES device comprising parallel electrodes and a network of four code-multiplexed sensors encoding 7-bit orthogonal Gold sequences. The four sensors are encoded with orthogonal codes, “1010110”, “0111111”, “0100010”, and “0011000” respectively. The resulting representative signals corresponding to each of the four individual sensors are illustrated in FIG. 19. The recorded signals from each sensor demonstrate the ability to generate distinct bipolar code signals from an electrode layout that is significantly simplified compared to the conventional coplanar electrode arrangement typically used to generate similar signal waveforms.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

What is claimed is:

1. A parallel electrode sensor for use with microfluidic devices, comprising:
a. a first electrode;
b. a second electrode;
c. a substrate having an upper surface, wherein the first electrode is deposited on the upper surface of the substrate; and
d. a microchannel comprising an interior surface, wherein the second electrode is deposited on the interior surface of the microchannel, the microchannel is oriented above and bonded to the substrate to form a fluidic channel so that the interior surface and the upper surface of the substrate are facing each other, placing the first electrode and the second electrode in parallel with each other.

2. The parallel electrode sensor of claim 1, wherein the first electrode comprises a first set of electrodes, wherein the upper surface of the substrate is lithographically treated to receive the deposit of the first electrodes.

3. The parallel electrode sensor of claim 2, wherein the substrate is lithographically treated to form a pattern.

4. The parallel electrode sensor of claim 3, wherein the substrate is lithographically treated by applying a photoresist before depositing the first set of electrodes.

5. The parallel electrode sensor of claim 3, wherein the pattern comprises an array.

6. The parallel electrode sensor of claim 2, wherein the first set of electrodes comprises a metallic layer deposited on the upper surface of the substrate.

7. The parallel electrode sensor of claim 1, wherein the microchannel is formed from a biocompatible polymer.

8. The parallel electrode sensor of claim 1, wherein the second electrode comprises a second set of electrodes and are deposited on the interior surface of the microchannel.

9. The parallel electrode sensor of claim 8, wherein the microchannel further comprises a ceiling and side walls.

10. The parallel electrode sensor of claim 8, wherein the microchannel further comprises a pattern to receive the second set of electrodes.

11. The parallel electrode sensor of claim 9, wherein the second set of electrodes is formed from a metallic layer deposited on the interior surface of the microchannel.

12. A method of forming a parallel electrode sensor for use with microfluidic devices, comprising:
a. lithographically patterning a first set of electrodes on an upper surface of a substrate;
b. depositing a second set of electrodes on an interior surface of a microchannel; and
c. bonding the microchannel with the substrate to form a fluidic channel so that the upper surface of the substrate and the interior surface of the microchannel face each other with the first set of electrodes and the second set of electrodes being in parallel relation to one another.

13. The method of claim 12, wherein depositing the second set of electrodes on the interior surface of the microchannel further comprises forming a microchannel from a biocompatible polymer, wherein the microchannel comprises a ceiling and side walls.

14. The method of claim 13, wherein the microchannel is formed from molding the biocompatible polymer using a mold, wherein the mold includes a pattern that is imprinted into the interior surface of the microchannel.

15. The method of claim 14, wherein depositing the second set of electrodes comprises depositing a metallic layer on the pattern of the microchannel.

16. The method of claim 15, wherein the depositing comprises sputtering.

17. The method of claim 15, wherein after depositing the metallic layer on the pattern, removing excess metallic layer from the interior surface not on the pattern to prevent short circuiting.

18. The method of claim 15, wherein lithographically patterning the first set of electrodes comprises applying another metallic layer on the upper surface of the substrate after a lithographic pattern is applied to the upper surface.

19. The method of claim 18, wherein the pattern of the upper surface of the substrate and the imprinted pattern of the microchannel substantially match one another.

20. The method of claim 14, wherein after forming the microchannel from the mold, providing an electric port into the microchannel.
